(19) United States
(12) Patent Application Publication
(10) Pub. No.: US 2003/0114409 A1
(43) Pub. Date: Jun. 19, 2003

(54) Facilitation of RNA interference

(76) Inventors: Craig C. Mello, Shrewsbury, MA (US); Chun-Chieh Chen, Taichung City (TW); Darryl Conte JR., Worcester, MA (US)

Correspondence Address: J. PETER FASSE, FISH & RICHARDSON P.C., 225 Franklin Street, Boston, MA, 02110-2804, US

(21) Appl. No.: 10/295,809

(22) Filed: Nov. 15, 2002

Related U.S. Application Data
(60) Provisional application No. 60/333,811, filed on Nov. 16, 2001.
(60) Provisional application No. 60/331,672, filed on Nov. 19, 2001.

(51) Int. Cl. A61K 48/00; C12N 15/85

(52) U.S. Cl. 514/44; 435/455; 435/375

(57) ABSTRACT

The present invention features compositions and methods to induce or enhance RNAi in cells, systems, and organisms using molecules that mediate RNAi in invertebrates such as C. elegans.

### CROSS-REFERENCE TO RELATED APPLICATIONS

The present application claims the benefit of the filing dates of U.S. Ser. No. 60/333,811, filed Nov. 16, 2002, and U.S. Ser. No. 60/331,672, filed Nov. 19, 2001. The contents of U.S. Ser. No. 60/333,811 and U.S. Ser. No. 60/331,672 are hereby incorporated by reference in the present application in their entirety.

### STATEMENT AS TO FEDERALLY SPONSORED RESEARCH

The work described below was funded, in part, by a grant from the federal government (GM5880). The government therefore has certain rights in the invention.

### TECHNICAL FIELD

This invention relates to RNA interference (RNAi).

### BACKGROUND

All eukaryotic organisms share similar mechanisms for information transfer from DNA to RNA to protein. RNA interference (RNAi) represents an efficient mechanism for inactivating this transfer process for a specific targeted gene. Targeting is mediated by the sequence of the RNA molecule introduced to the cell. Double-stranded RNA (dsRNA) can induce sequence-specific inhibition of gene function (genetic interference) in several organisms including the nematode, C. elegans (Fire et al., Nature 391:806-811, 1998), plants, trypanosomes, Drosophila, and planaria (Waterhouse et al., Proc. Natl. Acad. Sci. USA 94:13959-13964, 1998; Ngo et al., Proc. Natl. Acad. Sci. USA 95:14687-14692, 1998; Kennerdell and Carthew, Cell 95:1017-1026, 1998; Misquitta and Patterson, Proc. Natl. Acad. Sci. USA 96:1451-1456, 1999; Sanchez-Alvorado and Newmark, Proc. Natl. Acad. Sci. USA 96:5049-5054, 1999). The discovery that dsRNA can induce genetic interference in organisms from several distinct phyla suggests a conserved mechanism and perhaps a conserved physiological role for the interference process. Although several models of RNAi have been proposed (Baulcombe, Curr. Biol. 9:R599-R601,1999; Sharp, Genes & Dev. 13:139-141, 1999), the mechanisms of action of specific components of the pathway are not known.

### SUMMARY

The invention is based, in part, on the discovery that members of the C. elegans RNA-dependent RNA polymerase (RdRP) gene family are involved in, and can be essential for, RNAi. Thus, RdRP expression can be used to induce or enhance RNAi in cells, including mammalian cells (and, following on, in the systems and organisms in which those cells reside). The cells may lack RdRP expression, have some RdRP expression, or have robust RdRP expression. As described further below, the sequences encoding an RdRP polypeptide (or other polypeptide of the invention) can be fused to one or more sequences that encode heterologous polypeptides (e.g., a polypeptide that is detectable and thereby serves as a marker). Moreover, RdRP genes can be expressed in combination with one or more of the other genes of the RNAi system, such as Dicer, RDE-1, or RDE-4.

The invention also encompasses genetically engineered cells (e.g., mammalian and non-mammalian cells), cell lines (e.g., mammalian and non-mammalian cell lines), transgenic animals (e.g., non-human transgenic animals), and nucleic acid constructs that express an RdRP polypeptide (alone or in combination with other polypeptides (full-length or partial-length) that mediate RNAi). The compositions of the invention can be used, for example, to enhance the sensitivity to genetic interference induced by dsRNA in plants, vertebrate animals (including humans), non-human primates, other mammals, and invertebrates. For example, the invention features methods for inducing or enhancing RNAi in a cell by providing an RdRP polypeptide to the cell (as noted elsewhere, any construct used to provide the RdRP polypeptide and the genetically engineered cells that produce those polypeptides are also within the scope of the invention). The polypeptide can be provided by increasing the expression or activity of RdRP (e.g., EGO-1 or RRF-1) in the cell by, for example, introducing a construct that encodes the polypeptide in the cell, administering the polypeptide, or administering a therapeutic agent that mimics or increases the activity of an endogenous RdRP. The RdRP can be that of an invertebrate animal, such as C. elegans, and the nucleic acid sequence encoding the RdRP polypeptide can be operatively linked to a regulatory sequence (e.g., a mammalian regulatory sequence). In some embodiments, the cell can be a cell within a transgenic animal; these cells and transgenic animals are also within the scope of the invention. Various expression systems can be used, including those described further below, as can any mammalian cell type (e.g., stem cells (such as embryonic stem cells), hematopoietic cells, muscle cells (such as cardiac cells), endothelial cells (such as those found in the vascular system), or blood cells (such as lymphocytes).

As used herein, both “protein” and “polypeptide” mean any chain of amino acids, regardless of length or post-translational modification (e.g., glycosylation or phosphorylation). Thus, the term “RNAi pathway polypeptide” includes a full-length, naturally occurring RNAi pathway polypeptide such as EGO-1 protein or RRF-1 protein, as well as recombinantly or synthetically produced polypeptides that correspond to a full-length, naturally occurring EGO-1 protein, RRF-1 protein, or to particular domains or portions of a naturally occurring RNAi pathway protein.

An RNAi pathway component is a protein or nucleic acid that is involved in promoting dsRNA-mediated genetic interference. A nucleic acid component can be an RNA or DNA molecule. A mutation in a gene encoding an RNAi pathway component may decrease or increase RNAi pathway activity.

An RNAi pathway protein is a protein that is involved in promoting dsRNA-mediated genetic interference.

By “inhibited RNAi pathway” is meant decreased inhibitory activity of a dsRNA which results in at least two-fold lower inhibition by a dsRNA relative to its ability to cause inhibition in a wild type cell. Techniques for measuring RNAi pathway activity are described herein and are known in the art. The pathway can be inhibited by inhibiting a component of the pathway (e.g., EGO-1 or RRF-1) or mutating the component so that its function is reduced.

A “transgene” is any nucleic acid molecule that is inserted by artifice into a cell and that, after insertion, can become part of the genome of the organism that develops from the cell. The transgene may include a nucleic acid sequence (e.g., a gene) that is partly or entirely heterologous (i.e., foreign) to the transgenic cell or organism, or it can have a sequence (e.g., a gene sequence) that is homologous to an endogenous gene of the organism. The term “transgene” encompasses a nucleic acid molecule that encodes one or more RdRPs, or portions thereof, and, in some embodiments, additional sequences that encode proteins or nucleic acids involved in RNAi. These sequences can be partly or entirely heterologous to sequences in the cell or the transgenic animal in which they are expressed, or homologous to an endogenous gene of the transgenic animal (in which case, they can be designed to insert into the genome at a location that differs from that of the natural gene. A transgene can include one or more promoters and any other DNA, such as intronic sequence, necessary for expression of the selected nucleic acid sequence, all operably linked to the selected sequence, and may also (alternatively or in addition to the promoter) include an enhancer sequence. If more than one transgene is present, the transgenes may or may not be operably linked to each other. Any animal that can be produced by transgenic technology to express an RdRP polypeptide, or a portion thereof, is included in the invention, although mammals are preferred. Such mammals include non-human primates, sheep, goats, horses, cattle, pigs, rabbits, and rodents such as guinea pigs, hamsters, rats, gerbils, and mice.

A “transgenic cell” is a cell into which (or into an ancestor of which) has been introduced, by means of recombinant DNA techniques, a nucleic acid molecule or transgene encoding an RdRP such as EGO-1 or RRF-1. In some embodiments of the invention, additional RNAi genes are also introduced into a cell. A transgenic cell can be generated by, for example, transfection (including lipofection) or infection (with, e.g., a viral vector, such as a retroviral vector).

As used herein, the term “operably linked” means that a selected nucleic acid sequence, for example, a sequence encoding an RdRP or other polypeptide of the invention, is in proximity with a promoter (e.g., a constitutively active or a tissue-specific promoter), to allow the promoter to regulate expression of the selected nucleic acid sequence. In addition, the promoter is located upstream of the selected nucleic acid sequence in terms of the direction of transcription and translation.

By “promoter” is meant a nucleic acid sequence that is sufficient to direct transcription. A tissue-specific promoter effects expression of the selected nucleic acid sequence in specific cells, for example, hematopoietic cells or cells of a specific tissue within an animal (e.g. cardiac, muscle, or vascular tissue). The term also covers so-called “leaky” promoters, which regulate expression of a selected nucleic acid sequence primarily in one tissue, but cause expression in other tissues as well. Such promoters also may include additional DNA sequences, such as introns and enhancer sequences.

A “substantially pure” nucleic acid molecule or sequence is a nucleic acid molecule or sequence that is not immediately contiguous with both of the coding sequences with which it is immediately contiguous (one on the 5′ end and one on the 3′ end) in the naturally-occurring genome of the organism from which it is derived. The term therefore includes, for example, a recombinant DNA that is incorporated into a vector; into an autonomously replicating plasmid or virus; or into the genomic DNA of a prokaryote or eukaryote, or which exists as a separate molecule (e.g., a cDNA or a genomic DNA fragment produced by PCR or restriction endonuclease treatment) independent of other sequences. It also includes a recombinant DNA that is part of a hybrid gene encoding an additional polypeptide sequence.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

The compositions and methods described herein have several advantages. For example, the methods can provide enhanced RNAi in mammalian cells and in other cell types. Thus, RNAi can be more effectively used to inhibit gene expression. The expression of any gene can be inhibited; inhibiting genes whose expression is associated with uncontrolled cell growth (as occurs in cancer) can be beneficial in treating patients who have an uncontrolled cell growth (e.g. a tumor). The methods of the invention can also be used to inhibit the expression of viral genes. For example, one can inhibit genes essential for viral replication, packaging, or for any other viral life stages that occur within the host organism or human. This may be useful in treating patients who have a disease associated with a viral agent (e.g., AIDS patients, who are infected with a human immunodeficiency virus). The invention can also be used to inhibit expression of genes essential for the survival of pathogenic organisms that are susceptible to mechanisms involving RNAi, e.g., trypanosomes, nematodes, and other parasites such as Plasmodium falciparum.

The details of one or more embodiments of the invention are set forth in the accompanying drawings and the description below. Other features, objects, and advantages of the invention will be apparent from the description and drawings, and from the claims.

### DESCRIPTION OF DRAWINGS

FIGS. 1A-1E are diagrams showing the molecular cloning of rde-4 and rde-9 (A and C) Rescuing PCR products for rde-4 and rde-9 are illustrated using box/line diagrams to indicate the predicted exon/intron structure of the genes. The positions of genetic lesions associated with each mutant allele are indicated. (A) The double stranded RNA recognition motifs in RDE-4 are indicated by shading, and (B) are shown (SEQ ID NO:1) aligned with related motifs from PACT (Homo sapiens, AAC25672; SEQ ID NO:2), PRBP (Mus musculus, P97473; SEQ ID NO:3), CG6866 (Drosophila melanogaster, AE003640; SEQ ID NO:4), F21M12.9 (Arabidopsis thaliana, AAB60726; SEQ ID NO:5). Identities with RDE-4 are shaded in black, and identities among the related proteins are shaded in gray. (D) Portions of RDE-9 that contain lesions associated with each allele are aligned with corresponding regions from, C. elegans, plant and fungal homologs. The sequences shown are: RDE-9/RRF-1 (C. elegans, F26A3.8; SEQ ID NO:6), RRF-2 (C. elegans, M01G12.12; SEQ ID NO:7), EGO-1 (C. elegans, F26A3.3; SEQ ID NO:8), tomato(Lycopersicon esculentum, pir∥T30819; SEQ ID NO:10), tobacco (Nicotiana tabacum, pir∥T30828; SEQ ID NO:11), SGS2, (Arabidopsis thaliana, gb|AAF73959.1; SEQ ID NO:12), RRF-3 (C. elegans, F10B5.7; SEQ ID NO:13), pombe (Schizosaccharomyces pombe, pir∥T11660), QDE-1 (Neurospora crassa, gi|4803727; SEQ ID NO:14). (E) The four RdRP homologs, RDE-9/RRF-1, EGO-1, RRF-2 and RRF-3, were expressed as GFP fusions in the muscle under the control of the muscle-specific myo-3 promoter and assayed for the ability to rescue the RNAi defect of the rde-9(ne734) mutant. Rescue was assayed by monitoring muscle-twitching and paralysis caused by unc-22(RNAi). Rescuing transgenes gave rise to paralyzed twitchers (+), while non-rescuing transgenes failed to twitch (−). Rescue by the myo-3::gfp::rde-9 and myo-3::gfp::ego-1 constructs was comparable to rescue by the rde-9 genomic PCR product.

FIGS. 2A and 2B are photographs of immunoblots demonstrating that RDE-4 interacts with dsRNA during RNAi in C. elegans. (A and B) Immune complex precipitated using RDE-4 specific serum (+) or by using a control pre-immune serum (−) was prepared from wild-type and mutant C. elegans strains that were exposed to pos-1 dsRNA by feeding for 15 minutes or 2 days (as indicated). RNA extracted from each precipitate was heat-denatured and run on a denaturing (A) agarose-gel or (B) 15% acrylamide gel. The gels were blotted onto plus-charged nylon membranes and hybridized with sense or antisense RNA probes (as indicated). Radiolabeled size markers were used to indicate the approximate number of nucleotides (nt) in the migrating RNA species. Hybridization was performed overnight at 55° C. in 50% formamide, 2×SSC, 1% SDS, 5% dextran sulfate, 150 μg/ml Torula yeast RNA. The membrane was washed in (A) 0.2×SSC, 0.1% SDS, and in (B) 0.3×SSC, 0.1% SDS at 55° C.

FIGS. 3A and 3B are photographs of immunoblots demonstrating that RDE-4 and RDE-1 form a complex in vivo. (A and B) Cell lysates were prepared from an rde-1(ne300) mutant strain rescued with a transgene expressing both HA-tagged RDE-1 and FLAG-tagged RDE-4. As indicated above the lanes, total protein, or immune complex (IP) was electrophoresed on a denaturing polyacrylamide gel and probed with sera specific for HA::RDE-1, Flag::RDE-4, or RDE-4. In (A) RDE-4-specific serum, but not preimmune serum, immunoprecipitated both HA::RDE-1 and FLAG::RDE-4. In (B) IIA-specific serum, but not Normal IgG serum, immunoprecipitated both HA::RDE-1 and RDE-4.

FIGS. 4A-4D are photographs of Northern blots. Target-dependent accumulations of cRNA and siRNA species (A-D) Wild-type and rde-mutant populations were cultured with (+) or without (−) a gfp dsRNA trigger, and with or without a pes-10::gfp transgene. RNA prepared from each population (as indicated) was electrophoresed on (A and B) an agarose gel or (C and D) a 15% acrylamide gel. Each gel was blotted onto plus-charged nylon membrane and the membranes were hybridized sequentially with radiolabeled antisense and sense RNA probes (as indicated). (A and B) Between application of the probes, the blot was stripped to remove antisense probe. (C and D) Synthetic sense (s) and antisense (as) RNA oligos were used as hybridization controls (as indicated). Since no specific signal was detected, the antisense probe (C) was not stripped before hybridizing with the sense probe (D).

FIGS. 5A and B are micrographs and FIG. 5C is a bar graph. (A, B) Fluorescence micrographs of GFP expression after heat-shock in representative adult animals that carry (A) a heat-shock inducible GFP transgene (hsp16-2::gfp) or (B) both hsp16-2::gfp and a second constitutive pes-10::gfp transgene. Animals were either not exposed to gfp dsRNA (A and B, upper panels) or were continuously exposed to gfp dsRNA by feeding (A and B, lower panels). (C) Fractions of animals with silenced hsp16-2::gfp expression in the intestine and pharynx after the heat-shock (the strains and gfp dsRNA treatment are indicated).

FIG. 6 is a photograph of an immunoblot demonstrating siRNA accumulation during pos-1(RNAi). Wild-type (N2) and mutant strains, mut-7 and rde-1, were cultured for 48 hours on E. coli expressing pos-1 dsRNA. An equivalent N2 population was also cultured without dsRNA food (as indicated). RNA was extracted from adult populations and electrophoresed on a 15% acrylamide gel. Synthetic radiolabeled size markers (M) and pos-1 dsRNA processed in Drosophila embryo extracts were loaded as controls. The gel was blotted to membrane and hybridized with a radiolabeled sense pos-1 RNA probe. Only populations of animals actively engaged in silencing (N2 cultured on pos-1 dsRNA food) exhibit accumulation of an antisense siRNA species.

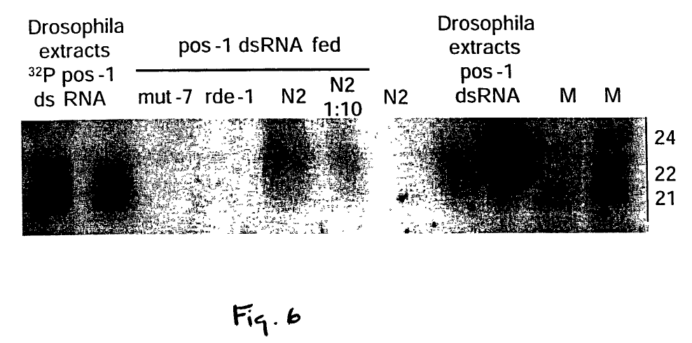

FIG. 7 is a table. The data included demonstrate that rde-9 is required for effective RNAi in somatic tissues, but not in the germline.

### DETAILED DESCRIPTION

Attempts to overexpress a gene (e.g., a transgene) often lead only to transient expression of the gene. Furthermore, the undesirable effect of “cosuppression” can occur. In this process, a corresponding endogenous copy of the transgene becomes inactivated. In some cases, transgene silencing leads to problems with the commercial or therapeutic application of transgenic technology.

RNAi is a post-transcriptional gene silencing (PTGS) mechanism that is triggered after double-stranded RNA (dsRNA) is introduced into a cell. RNAi has been reported to work in numerous animal systems, including Drosophila embryos and tissue culture, Xenopus oocytes, mouse embryos and, significantly, in human tissue culture (Cogoni and Macino, 2000, Proc. Nat. Acad. Sci. USA 94:10233-10238). Post-transcriptional gene silencing can also occur across kingdoms (Curr. Opin. Genet. Dev. 10:638-643; Oelgeschlager et al., 2000, Nature, 15:757-763; Wianny and Zernicka-Goetz, 2000, Nat. Cell Biol. 2:70-75; Elbashir et al., 2001, Nature 411:494-498). However, RNAi in these other systems is much less efficient than in C. elegans. The absence of cellular RNA-dependent RNA polymerase in the genomes of other animals may explain why RNAi does not work as efficiently in these systems. The present invention encompasses compositions and methods to induce or enhance RNAi in animals and humans using molecules that normally mediate RNAi in invertebrates such as C. elegans.

Previous work in fungi and plants suggested that a phenomenon called co-suppression requires RdRP related genes. In co-suppression, the addition of an extra copy of a gene via a transgene leads to silencing of both the transgene and the endogenous cellular copy of the gene. This phenomenon is thought to involve: 1) expression of the transgene derived mRNA, 2) recognition of this mRNA as foreign, 3) copying of the mRNA via RNA dependent RNA polymerase to produce dsRNA, and 4) processing of dsRNA into siRNA that target mRNA destruction (Cogoni and Macino, 2000, supra). In RNAi, the inducing sequence is already a dsRNA and so there is no reason to expect that RdRP would need to act in this mechanism. Essentially, RNAi would initiate the co-suppression process downstream of RdRP at, for example, step 4 (described above). Thus, one would not expect that an RdRP enzyme would be required for RNAi. Certainly, RdRP would not be expected to be required when a constant source of dsRNA is provided as through the action of DNA dependent polymerase.

A C. elegans family of genes has been identified as being essential for RNAi. These genes encode an RNA-dependent RNA polymerase enzymatic activity (RdRP) that may amplify the double-stranded RNA (dsRNA) or the small interfering RNAs (siRNAs) that mediate RNAi. Members of this gene family are not present in other higher animals, including Drosophila and vertebrates, where RNAi has been shown to occur, but with reduced efficiency relative to C. elegans. The lack of RdRP activity can explain the relatively low efficiency of RNAi in these other systems. Therefore, expressing RdRP activity in organisms lacking this activity leads to significantly enhanced RNAi.

In some embodiments of the invention, additional components of the C. elegans RNAi system (i.e., genes or gene products that are necessary for RNAi) are expressed in cells, e.g., mammalian cells, using RdRP encoding genes. The additional components of the proteins that mediate RNAi in the C. elegans systems that can be used include RDE-1, RDE-4, and DCR-1.

In the Examples below, it is shown that a C. elegans RdRP family member, RRF-1, is essential for RNAi in the somatic tissues of C. elegans. It is also shown that mutations in RRF-1 can be rescued by expressing the normally germ-line restricted, RdRP family member EGO-1, in the somatic tissues of the rrf-1 deficient animals. Thus, EGO-1 and RRF-1 are functionally equivalent, but are expressed in different tissues in C. elegans. Two other homologues of RRF-1 exist in C. elegans, but have not been demonstrated to compensate for RRF-1, and thus may lack important functional domains required for RNAi.

It was not previously known that an amplification system was necessary for RNAi to work efficiently. Large amounts of dsRNA are delivered in RNAi assays and thus sufficient quantities of siRNA might be expected to be processed directly from this “trigger” dsRNA. siRNAs derived directly from the trigger dsRNA could then mediate interference without a need for amplification. Instead, without RdRP activity, no RNAi is observed, even when dsRNA is continuously synthesized through the action of DNA-dependent RNA polymerase II. Without limiting the invention to compositions that work by any particular mechanism, it appears that RdRP-derived dsRNA is a more efficient inducer of RNAi than is exogenous dsRNA or dsRNA derived from a DNA template. The present inventors have recognized the importance of RdRP-dependent amplification and use this class of RNA synthesizing enzyme to induce efficient RNAi in other systems.

Methods of Expressing RNAi Pathway Proteins

Polypeptides, including those that include a full-length RNAi pathway protein such as an RdRP, as well as fragments thereof (e.g., fragments of an RNAi pathway protein corresponding to a functional domain) are also within the scope of the invention. Also within the invention are fusion proteins in which a portion (e.g., one or more domains) of an RdRP is fused to an unrelated protein or polypeptide (i.e., a fusion partner) to create a fusion protein. The fusion partner can be a moiety selected to facilitate purification, detection, or solubilization, or to provide some other function. For example, RdRP polypeptides can be fused to Rde-1, Rde-4, Dcr-1, domains within these polypeptides, or full- or partial-length homologous polypeptides from other organisms.

Fusion proteins are generally produced by expressing a hybrid gene in which a nucleotide sequence encoding all or a portion of an RNAi pathway protein is joined, in-frame, to a nucleotide sequence encoding the fusion partner. Fusion partners include, but are not limited to, the constant region of an immunoglobulin (IgFc). A fusion protein in which an RNAi pathway polypeptide is fused to IgFc can be more stable and have a longer half-life in the body than the polypeptide on its own.

In general, RNAi pathway proteins (e.g., RdRPs such as EGO-1 and RRF-1, RDE-1, and RDE-4) can be produced by transformation (transfection, transduction, or infection) of a host cell with all or part of an RNAi pathway protein-encoding DNA fragment (e.g., one of the cDNAs described herein) in a suitable expression vehicle. Suitable expression vehicles include: plasmids, viral particles, and phage. For insect cells, baculovirus expression vectors are suitable. The entire expression vehicle, or a part thereof, can be integrated into the host cell genome. In some circumstances, it is desirable to employ an inducible expression vector, for example, the LACSWITCH™ Inducible Expression System (Stratagene; LaJolla, Calif.).

Those skilled in the field of molecular biology will understand that any of a wide variety of expression systems can be used to provide the recombinant protein. The precise host cell used is not critical to the invention. The RNAi pathway protein can be produced in a prokaryotic host (e.g., E. coli or B. subtilis) or in a eukaryotic host (e.g., Saccharomyces or Pichia; mammalian cells, e.g., COS, NIH 3T3 CHO, BHK, 293, or HeLa cells; or insect cells). Proteins and polypeptides can also be produced in plant cells. For plant cells, viral expression vectors (e.g., cauliflower mosaic virus and tobacco mosaic virus) and plasmid expression vectors (e.g., Ti plasmid) are suitable. Such cells are available from a wide range of sources (e.g., the American Type Culture Collection, Manassas, Va.; see also, e.g., Ausubel et al., John Wiley & Sons, New York, 1994). The methods of transformation or transfection and the choice of expression vehicle will depend on the host system selected. Transformation and transfection methods are described, for example, in Ausubel et al., supra; expression vehicles may be chosen from those provided, for example, in Cloning Vectors: A Laboratory Manual (P. H. Pouwels et al., 1985, Supp. 1987).

The host cells harboring the expression vehicle can be cultured in conventional nutrient media adapted as needed for activation of a chosen gene, repression of a chosen gene, selection of transformants, or amplification of a chosen gene.

One useful expression system is the mouse 3T3 fibroblast host cell transfected with a pMAMneo expression vector (Clontech, Palo Alto, Calif.). pMAMneo provides an RSV-LTR enhancer linked to a dexamethasone-inducible MMTV-LTR promotor, an SV40 origin of replication which allows replication in mammalian systems, a selectable neomycin gene, and SV40 splicing and polyadenylation sites. DNA encoding an RNAi pathway protein is inserted into the pMAMneo vector in an orientation designed to allow expression. The recombinant RNAi pathway protein is isolated as described herein. Other host cells that can be used in conjunction with the pMAMneo expression vehicle include COS cells and CHO cells (ATCC Accession Nos. CRL 1650 and CCL 61, respectively).

RNAi pathway polypeptides can be produced as fusion proteins. For example, the expression vector pUR278 (Ruther et al., EMBO J. 2:1791, 1983) can be used to create lacZ fusion proteins. The pGEX vectors can be used to express foreign polypeptides as fusion proteins with glutathione S-transferase (GST). In general, such fusion proteins are soluble and can be easily purified from lysed cells by adsorption to glutathione-agarose beads followed by elution in the presence of free glutathione. The pGEX vectors are designed to include thrombin or factor Xa protease cleavage sites so that the cloned target gene product can be released from the GST moiety.

In an insect cell expression system, Autographa californica nuclear polyhedrosis virus (AcNPV), which grows in Spodoptera frugiperda cells, is used as a vector to express foreign genes. An RNAi pathway protein coding sequence can be cloned individually into non-essential regions (for example the polyhedrin gene) of the virus and placed under control of an AcNPV promoter, for example, the polyhedrin promoter. Successful insertion of a gene encoding an RNAi pathway polypeptide or protein will result in inactivation of the polyhedrin gene and production of non-occluded recombinant virus (i.e., virus lacking the proteinaceous coat encoded by the polyhedrin gene). These recombinant viruses are then used to infect Spodoptera frugiperda cells in which the inserted gene is expressed (see, e.g., Smith et al., J. Virol. 46:584, 1983; Smith, U.S. Pat. No. 4,215,051).

In mammalian host cells, a number of viral-based expression systems can be utilized. When an adenovirus is used as an expression vector, the RNAi pathway protein nucleic acid sequence can be ligated to an adenovirus transcription/translation control complex, for example, the late promoter and tripartite leader sequence. This chimeric gene can then be inserted into the adenovirus genome by in vitro or in vivo recombination. Insertion into a non-essential region of the viral genome (e.g., region E1 or E3) will result in a recombinant virus that is viable and capable of expressing an RNAi pathway gene product in infected hosts (see, e.g., Logan, Proc. Natl. Acad. Sci. USA 81:3655, 1984).

Specific initiation signals may be required for efficient translation of inserted nucleic acid sequences. These signals include the ATG initiation codon and adjacent sequences. In cases where an entire native RNAi pathway protein gene or cDNA, including its own initiation codon and adjacent sequences, is inserted into the appropriate expression vector, no additional translational control signals may be needed. In other cases, exogenous translational control signals, including, perhaps, the ATG initiation codon, must be provided. Furthermore, the initiation codon must be in phase with the reading frame of the desired coding sequence to ensure translation of the entire insert. These exogenous translational control signals and initiation codons can be of a variety of origins, both natural and synthetic. The efficiency of expression may be enhanced by the inclusion of appropriate transcription enhancer elements, transcription terminators (Bittner et al., Methods in Enzymol. 153:516, 1987).

RNAi pathway polypeptides (e.g., RdRP) can be expressed directly or as a fusion with a heterologous polypeptide, such as a signal sequence or other polypeptide having a specific cleavage site at the N-and/or C-terminus of the mature protein or polypeptide. Included within the scope of this invention are RNAi pathway polypeptides with a heterologous signal sequence. The heterologous signal sequence selected should be one that is recognized and processed, for example, cleaved by a signal peptidase, by the host cell. For prokaryotic host cells a prokaryotic signal sequence is selected, for example, from the group of the alkaline phosphatase, penicillinase, lpp, or heat-stable enterotoxin II leaders. For yeast secretion, yeast invertase, alpha factor, or acid phosphatase leaders may be selected. In mammalian cells, it is generally desirable to select a mammalian signal sequences.

A host cell can be chosen that modulates the expression of the inserted sequences, or modifies and processes the gene product in a specific, desired fashion. Such modifications (e.g., glycosylation) and processing (e.g., cleavage) of protein products may be important for the function of the protein. Different host cells have characteristic and specific mechanisms for the post-translational processing and modification of proteins and gene products. Appropriate cell lines or host systems can be chosen to ensure the correct modification and processing of the foreign protein expressed. To this end, eukaryotic host cells that possess the cellular machinery for proper processing of the primary transcript, glycosylation, and phosphorylation of the gene product can be used. Such mammalian host cells include, but are not limited to, CHO, VERO, BHK, HeLa, COS, MDCK, 293, 3T3, WI38, and in particular, choroid plexus cell lines.

Alternatively, an RNAi pathway protein can be produced by a stably-transfected mammalian cell line. A number of vectors suitable for stable transfection of mammalian cells are available to the public, see, e.g., Pouwels et al. (supra); methods for constructing such cell lines are also publicly available, e.g., in Ausubel et al. (supra). In one example, CDNA encoding an RNAi pathway protein (e.g., EGO-1, RRF-1, DCE, RDE-1, or RDE-4) is cloned into an expression vector that includes the dihydrofolate reductase (DHFR) gene. Integration of the plasmid and, therefore, the RNAi pathway protein-encoding gene into the host cell chromosome is selected for by including 0.01-300 μM methotrexate in the cell culture medium (as described in Ausubel et al., supra). This dominant selection can be accomplished in most cell types.

Recombinant protein expression can be increased by DHFR-mediated amplification of the transfected gene. Methods for selecting cell lines bearing gene amplifications are also described in Ausubel et al. (supra); such methods generally involve extended culture in medium containing gradually increasing levels of methotrexate. DHFR-containing expression vectors commonly used for this purpose include pCVSEII-DHFR and pAdD26SV(A) (described in Ausubel et al., supra). Any of the host cells described above or, preferably, a DHFR-deficient CHO cell line (e.g., CHO DHFR-cells, ATCC Accession No. CRL 9096) are among the host cells preferred for DHFR selection of a stably-transfected cell line or DHFR-mediated gene amplification.

A number of other selection systems can be used, including, but not limited to, the herpes simplex virus thymidine kinase, hypoxanthine-guanine phosphoribosyl-transferase, and adenine phosphoribosyltransferase genes can be employed in tk, hgprt, or aprt cells, respectively. In addition, gpt, which confers resistance to mycophenolic acid (Mulligan et al., Proc. Natl. Acad. Sci. USA, 78:2072, 1981); neo, which confers resistance to the aminoglycoside G-418 (Colberre-Garapin et al., J. Mol. Biol., 150:1, 1981); and hygro, which confers resistance to hygromycin (Santerre et al., Gene 30:147, 1981), can be used.

Alternatively, any fusion protein can be readily purified by utilizing an antibody specific for the fusion protein being expressed. For example, a system described in Janknecht et al. (Proc. Natl. Acad. Sci. USA 88:8972, 1981) allows for the ready purification of non-denatured fusion proteins expressed in human cell lines. In this system, the gene of interest is subcloned into a vaccinia recombination plasmid such that the gene's open reading frame is translationally fused to an amino-terminal tag consisting of six histidine residues. Extracts from cells infected with recombinant vaccinia virus are loaded onto Ni2⁺ nitriloacetic acid-agarose columns, and histidine-tagged proteins are selectively eluted with imidazole-containing buffers.

Alternatively, an RNAi pathway protein or a portion thereof, can be fused to an immunoglobulin Fc domain. Such a fusion protein can be readily purified using a protein A column.

Ectopic Expression of an RNAi Pathway Gene

Ectopic expression of an RdRP such as EGO-1 or RRF-1 (i.e., expression of an RdRP gene or other RNAi pathway gene in a cell where it is not normally expressed or at a time when it is not normally expressed) can be used to enhance RNAi. Ectopic expression is useful to, for example, decrease undesirable expression of a gene in a cell (e.g., a plant or an animal cell, including invertebrate cells, such as those in nematodes and Drosophila, and vertebrate cells, such as those in a mouse or a human).

Ectopic expression of an RNAi pathway gene (e.g., ego-1, rrf-1, rde-1, or rde-4) can be used to activate the RNAi pathway. In some cases, targeting can be used to activate the pathway in specific cell types, such as tumor cells or virally infected cells. For example, a non-viral RNAi pathway gene construct can be targeted in vivo to specific tissues or organs (e.g., the liver or muscle) in patients. Examples of delivery systems for targeting such constructs include receptor mediated endocytosis, liposome encapsulation (described infra), or direct insertion of non-viral expression vectors.

Successful in vivo gene transfer has been achieved with the injection of DNA as, for example, a linear construct or a circular plasmid. The DNA can be encapsulated in liposomes (as described, for example, by Ledley, Human Gene Therapy 6:1129-1144, 1995 or Farhood et al., Ann. NY Acad. Sci. 716:23-35, 1994). Targeted gene transfer has been shown to occur using such methods. For example, intratracheal administration of cationic lipid-DNA complexes was shown to effect gene transfer and expression in the epithelial cells lining the bronchus (Brigham et al., Am. J. Respir. Cell Mol. Biol. 8:209-213, 1993; Canonico et al., Am. J. Respir. Cell Mol. Biol. 10:24-29, 1994). Expression in pulmonary tissues and the endothelium was reported after intravenous injection of the complexes (Brigham et al., supra; see also Zhu et al., Science 261:209-211, 1993; Stewart et al., Human Gene Therapy 3:267-275, 1992; Nabel et al., Human Gene Therapy 3:649-656, 1992; and Canonico et al., J. Appl. Physiol. 77:415-419, 1994). An expression cassette for an RNAi pathway sequence in linear, plasmid, or viral DNA forms can be condensed through ionic interactions with the cationic lipid to form a particulate complex for in vivo delivery (Stewart et al., Human Gene Therapy 3:267-275, 1992).

Other liposome formulations, for example, proteoliposomes which contain viral envelope receptor proteins (e.g., virosomes), have been found to effectively deliver genes into hepatocytes and kidney cells after direct injection (Nicolau et al., Proc. Natl. Acad. Sci. USA 80:1068-1072, 1993); Kaneda et al., Science 243:375-378, 1989); Mannino et al., Biotechniques 6:682, 1988); and Tomita et al., Biochem. Biophys. Res. Comm. 186:129-134, 1992).

Direct injection can also be used to administer an RNAi pathway nucleic acid sequence in a DNA expression vector (e.g., into the muscle, liver, or other tissue) either as a solution or as a calcium phosphate precipitate (Wolff et al., Science 247:1465-1468, 1990; Ascadi et al., The New Biologist 3:71-81, 1991; and Benvenisty et al., Proc. Natl. Acad. Sci. USA 83:9551-9555, 1986).

Transgenic Animals

Engineered RdRPs can be expressed in transgenic animals. RNAi is increased in these animals and thus there is decreased expression of targeted sequences in these animals. These animals represent a model system for the study of disorders that are caused by, or exacerbated by, underexpression of nucleic acids or polypeptides targeted for destruction by the RNAi system, and for the development of therapeutic agents that modulate the expression or activity of nucleic acids or polypeptides targeted for destruction.

Transgenic animals can be farm animals (pigs, goats, sheep, cows, horses, rabbits, and the like), rodents (such as rats, guinea pigs, and mice), non-human primates (for example, baboons, monkeys, and chimpanzees), and domestic animals (for example, dogs and cats). Methods for generating transgenic animals are well known to those of ordinary skill in the art (see also, below).

Regulatory Sequences

The expression of the RdRP (e.g., EGO-1 and RRF-1) and other RNAi genes used in the invention is driven by a regulatory sequence. The term regulatory sequence includes promoters, enhancers and other expression control elements. The appropriate regulatory sequence depends on such factors as the future use of the transgenic animal, and the level of expression of the desired RNA precursor. A person skilled in the art would be able to choose the appropriate regulatory sequence. For example, the transgenic animals described herein can be used to enhance RNAi in a particular cell type, for example, a hematopoietic cell. In this case, a regulatory sequence that drives expression of the transgene ubiquitously, or a hematopoietic-specific regulatory sequence that expresses the transgene only in hematopoietic cells, can be used. Expression of the RdRP in a hematopoietic cell means that the cell is now susceptible to enhanced RNAi. Examples of various regulatory sequences are described below.

The regulatory sequence can be inducible or constitutive. Suitable constitutive regulatory sequences include the regulatory sequence of a housekeeping gene such as the α-actin regulatory sequence, or regulatory sequences may be of viral origin such as regulatory sequences derived from mouse mammary tumor virus (MMTV) or cytomegalovirus (CMV).

Alternatively, the regulatory sequence can direct transgene expression in specific organs or cell types (see, e.g., Lasko et al., Proc. Natl. Acad. Sci. USA 89:6232, 1992). Tissue-specific regulatory sequences are known in the art including the albumin regulatory sequence for liver (Pinkert et al., Genes Dev. 1:268-276, 1987); the endothelin regulatory sequence for endothelial cells (Lee, J. Biol. Chem. 265:10446-50, 1990); the keratin regulatory sequence for epidermis; the myosin light chain-2 regulatory sequence for heart (Lee et al., J. Biol. Chem., 267:15875-85, 1992), and the insulin regulatory sequence for pancreas (Bucchini et al, Proc. Natl. Acad. Sci USA., 83:2511-2515, 1986), or the vav regulatory sequence for hematopoietic cells (Oligvy et al., Proc. Natl. Acad. Sci., USA, 96:14943-14948, 1999). Another suitable regulatory sequence, which directs constitutive expression of transgenes in cells of hematopoietic origin, is the murine MHC class I regulatory sequence (Morello et al, EMBO J. 5:1877-1882, 1986). Since MHC expression is induced by cytokines, expression of a test gene operably linked to this regulatory sequence can be upregulated in the presence of cytokines.

In addition, expression of the transgene can be precisely regulated, for example, by using an inducible regulatory sequence and expression systems such as a regulatory sequence that is sensitive to certain physiological regulators (e.g., circulating glucose levels or hormones) (Docherty et al. FASEB J. 8:20-24, 1994). Such inducible expression systems, suitable for the control of transgene expression in mammals such as mice, include regulation by ecdysone, by estrogen, progesterone, tetracycline, chemical inducers of dimerization, and isopropyl-beta-D-1-thiogalactopyranoside (IPTG)(collectively referred to as “the regulatory molecule”). Each of these expression systems is well described in the literature and permits expression of the transgene throughout the animal in a fashion controlled by the presence or absence of the regulatory molecule. For a review of inducible expression systems, see, e.g., Mills, Genes Devel., 15:1461-1467, 2001), and references cited therein.

Procedures for Making Transgenic, Non-Human Animals

A number of methods have been used to obtain transgenic, non-human animals, which are animals that have gained an additional gene by the introduction of a transgene into their cells (e.g., both the somatic and germ cells), or into an ancestor's germ line.

Methods for generating transgenic animals include introducing the transgene into the germ line of the animal. One method is by microinjection of a gene construct into the pronucleus of an early stage embryo (e.g., before the four-cell stage; Wagner et al., Proc. Natl. Acad. Sci., USA 78:5016, 1981; Brinster et al., Proc. Natl. Acad. Sci., USA, 82:4438, 1985). Alternatively, the transgene can be introduced into the pronucleus by retroviral infection. A detailed procedure for producing such transgenic mice has been described (see e.g., Hogan et al., Manipulating the Mouse Embryo, Cold Spring Harbor Laboratory Press, Cold Spring Harbor, N.Y., 1986; U.S. Pat. No. 5,175,383). This procedure has also been adapted for other animal species (e.g., Hammer et al., Nature 315:680, 1985); Murray et al., Reprod. Fert. Devl. 1:147, 1989); Pursel et al., Vet. Immunol. Histopath. 17:303, 1987; Rexroad et al., J. Reprod. Fert. 41 (suppl):119, 1990; Rexroad et al., Molec. Reprod. Devl. 1:164, 1989); Simons et al., BioTechnology 6:179, 1988; Vize et al., J. Cell. Sci. 90:295, 1988); and Wagner, J. Cell. Biochem. 13B (suppl):164, 1989).

In brief, the procedure involves introducing the transgene into an animal by microinjecting the construct into the pronuclei of the fertilized mammalian egg(s) to cause one or more copies of the transgene to be retained in the cells of the developing mammal(s). Following introduction of the transgene construct into the fertilized egg, the egg may be incubated in vitro for varying amounts of time, or reimplanted a surrogate host, or both. One common method is to incubate the embryos in vitro for about 1-7 days, depending on the species, and then reimplant them into the surrogate host. The presence of the transgene in the progeny of the transgenically manipulated embryos can be tested by Southern blot analysis of a segment of tissue. Another method for producing germ-line transgenic animals is through the use of embryonic stem (ES) cells. The gene construct can be introduced into embryonic stem cells by homologous recombination (Thomas et al., Cell 51:503, 1987; Capecchi, Science 244:1288, 1989; Joyner et al., Nature 338:153, 1989) in a transcriptionally active region of the genome. A suitable construct can also be introduced into embryonic stem cells by DNA-mediated transfection, such as by electroporation (Ausubel et al., Current Protocols in Molecular Biology, John Wiley & Sons, 1987). Detailed procedures for culturing embryonic stem cells (e.g., ES-D3, ATCC# CCL-1934, ES-E14TG2a, ATCC# CCL-1821, American Type Culture Collection, Manassas, Va.) and methods of making transgenic animals from embryonic stem cells can be found in Teratocarcinomas and Embryonic Stem Cells, A Practical Approach, ed. E. J. Robertson (IRL Press, 1987). In brief, the ES cells are obtained from pre-implantation embryos cultured in vitro (Evans et al, Nature 292:154-156, 1981). Transgenes can be efficiently introduced into ES cells by DNA transfection or by retrovirus-mediated transduction. The resulting transformed ES cells can thereafter be combined with blastocysts from a non-human animal. The ES cells colonize the embryo and contribute to the germ line of the resulting chimeric animal.

In the above methods, the transgene can be introduced as a linear construct, a circular plasmid, or a viral vector, which can be incorporated and inherited as a transgene integrated into the host genome. The transgene can also be constructed to permit it to be inherited as an extrachromosomal plasmid (Gassmann et al, Proc. Natl. Acad. Sci., USA, 92:1292., 1995). A plasmid is a DNA molecule that can replicate autonomously in a host.

The transgenic, non-human animals can also be obtained by infecting or transfecting cells either in vivo (e.g., direct injection), ex vivo (e.g., infecting the cells outside the host and later reimplanting), or in vitro (e.g., infecting the cells outside host), for example, with a recombinant viral vector carrying a gene encoding the RdRP (and in some embodiments, additional RNAi genes such as rde-1 and rde-4). Examples of suitable viral vectors include recombinant retroviral vectors (Valerio et al., Gene 84:419, 1989; Scharfman et al., Proc. Natl. Acad. Sci., USA, 88:462, 1991; Miller and Buttimore, Mol. Cell. Biol., 6:2895, 1986), recombinant adenoviral vectors (Freidman et al., Mol. Cell. Biol. 6:3791, 1986; Levrero et al., Gene 101:195, 1991), and recombinant Herpes simplex viral vectors (Fink et al., Human Gene Therapy 3:11, 1992).

Other approaches include insertion of transgenes encoding an RdRP or other RNAi sequence used in the invention into viral vectors including recombinant adenovirus, adeno-associated virus, and herpes simplex virus-1, or recombinant bacterial or eukaryotic plasmids. Viral vectors transfect cells directly. Other approaches include delivering the transgenes, in the form of plasmid DNA, with the help of, for example, cationic liposomes (lipofectin) or derivatized (e.g. antibody conjugated), polylysine conjugates, gramacidin S, artificial viral envelopes or other such intracellular carriers, as well as direct injection of the transgene construct or CaPO₄ precipitation carried out in vivo.

Retrovirus vectors and adeno-associated virus vectors can be used as a recombinant gene delivery system for the transfer of exogenous genes in vivo. These vectors provide efficient delivery of genes into cells, and the transferred nucleic acids are stably integrated into the chromosomal DNA of the host. The development of specialized cell lines (termed “packaging cells”) which produce only replication-defective retroviruses has increased the utility of retroviruses for gene therapy, and defective retroviruses are characterized for use in gene transfer for gene therapy purposes (for a review see Miller, Blood 76:271, 1990). A replication defective retrovirus can be packaged into virions that can be used to infect a target cell through the use of a helper virus by standard techniques. Protocols for producing recombinant retroviruses and for infecting cells in vitro or in vivo with such viruses can be found in Current Protocols in Molecular Biology (Ausubel et al. eds., Greene Publishing Associates, 1989, Sections 9.10-9.14) and other standard laboratory manuals.

Examples of suitable retroviruses include pLJ, pZIP, pWE and pEM which are known to those skilled in the art. Examples of suitable packaging virus lines for preparing both ecotropic and amphotropic retroviral systems include Psi-Crip, Psi-Cre, Psi-2 and Psi-Am. Retroviruses have been used to introduce a variety of genes into many different cell types, including epithelial cells, in vitro and/or in vivo (see for example Eglitis et al., Science 230:1395-1398, 1985; Danos and Mulligan, Proc. Natl. Acad. Sci. USA 85:6460-6464, 1988; Wilson et al., Proc. Natl. Acad. Sci. USA 85:3014-3018, 1988; Armentano et al., Proc. Natl. Acad. Sci. USA 87:6141-6145, 1990; Huber et al. Proc. Natl. Acad. Sci. USA 88:8039-8043, 1991; Ferry et al., Proc. Natl. Acad. Sci. USA 88:8377-8381, 1991; Chowdhury et al., Science 254:1802-1805, 1991; van Beusechem et al., Proc. Natl. Acad. Sci. USA 89:7640-7644, 1992; Kay et al., Human Gene Therapy 3:641-647, 1992; Dai et al., Proc. Natl. Acad. Sci. USA 89:10892-10895, 1992; Hwu et al. J. Immunol. 150:4104-4115, 1993; U.S. Pat. No. 4,868,116; U.S. Pat. No. 4,980,286; PCT Application WO 89/07136; PCT Application WO 89/02468; PCT Application WO 89/05345; and PCT Application WO 92/07573).

In another example, recombinant retroviral vectors capable of transducing and expressing genes inserted into the genome of a cell can be produced by transfecting the recombinant retroviral genome into suitable packaging cell lines such as PA317 and Psi-CRIP (Cornette et al., Human Gene Therapy 2:5-10, 1991; Cone et al, Proc. Natl. Acad. Sci., USA, 81:6349, 1984). Recombinant adenoviral vectors can be used to infect a wide variety of cells and tissues in susceptible hosts (e.g., rat, hamster, dog, and chimpanzee) (Hsu et al., J. Infectious Disease, 166:769,1992), and also have the advantage of not requiring mitotically active cells for infection.

Another viral gene delivery system useful in the present invention utilizes adenovirus-derived vectors. The genome of an adenovirus can be manipulated such that it encodes and expresses a gene product of interest but is inactivated in terms of its ability to replicate in a normal lytic viral life cycle. See, for example, Berkner et al.BioTechniques 6:616, 1988; Rosenfeld et al., Science 252:431-434, 1991; and Rosenfeld et al., Cell 68:143-155, 1992. Suitable adenoviral vectors derived from the adenovirus strain Ad type 5 dl324 or other strains of adenovirus (e.g., Ad2, Ad3, Ad7 etc.) are known to those skilled in the art. Recombinant adenoviruses can be advantageous in certain circumstances in that they are not capable of infecting nondividing cells and can be used to infect a wide variety of cell types, including epithelial cells (Rosenfeld et al., supra, 1992). Furthermore, the virus particle is relatively stable and amenable to purification and concentration, and as above, can be modified to affect the spectrum of infectivity. Additionally, introduced adenoviral DNA (and foreign DNA contained therein) is not integrated into the genome of a host cell but remains episomal, thereby avoiding potential problems that can occur as a result of insertional mutagenesis in situ where introduced DNA becomes integrated into the host genome (e.g., retroviral DNA). Moreover, the carrying capacity of the adenoviral genome for foreign DNA is large (up to 8 kilobases) relative to other gene delivery vectors (Berkner et al., supra; Haj-Ahmand and Graham, J. Virol. 57:267, 1986).

Yet another viral vector system useful for delivery of the subject transgenes is the adeno-associated virus (AAV). Adeno-associated virus is a naturally occurring defective virus that requires another virus, such as an adenovirus or a herpes virus, as a helper virus for efficient replication and a productive life cycle. For a review, see Muzyczka et al.(Curr. Topics in Micro. and Immunol. 158:97-129, 1992). It is also one of the few viruses that may integrate its DNA into non-dividing cells, and exhibits a high frequency of stable integration (see, for example, Flotte et al., Am. J. Respir. Cell. Mol. Biol. 7:349-356, 1992; Samulski et al., J. Virol. 63:3822-3828, 1989; and McLaughlin et al., J. Virol. 62:1963-1973, 1983). Vectors containing as little as 300 base pairs of AAV can be packaged and can integrate. Space for exogenous DNA is limited to about 4.5 kb. An AAV vector such as that described in Tratschin et al. (Mol. Cell. Biol. 5:3251-3260, 1985) can be used to introduce DNA into cells. A variety of nucleic acids have been introduced into different cell types using AAV vectors (see, for example, Hermonat et al., Proc. Natl. Acad. Sci. USA 81:6466-6470, 1984; Tratschin et al., Mol. Cell. Biol. 4:2072-2081, 1985; Wondisford et al. Mol. Endocrinol. 2:32-39, 1988; Tratschin et al., J. Virol. 51:611-619, 1984; and Flotte et al., J Biol. Chem. 268:3781-3790, 1993).

In addition to viral transfer methods, such as those described above, non-viral methods can also be employed to cause expression of an RdRP such as RRF-1 or EGO-1 in the tissue of an animal. Most non-viral methods of gene transfer rely on normal mechanisms used by mammalian cells for the uptake and intracellular transport of macromolecules. In preferred embodiments, non-viral gene delivery systems of the present invention rely on endocytic pathways for the uptake of the subject gene of the invention by the targeted cell. Exemplary gene delivery systems of this type include liposomal derived systems, poly-lysine conjugates, and artificial viral envelopes. Other embodiments include plasmid injection systems such as are described in Meuli et al., J. Invest. Dermatol., 116:131-135, 2001; Cohen et al., Gene Ther., 7:1896-1905, 2000; and Tam et al., (2000) Gene Ther., 7:1867-74, 2000.

In a representative embodiment, a gene encoding an engineered RdRP can be entrapped in liposomes bearing positive charges on their surface (e.g., lipofectins) and (optionally) which are tagged with antibodies against cell surface antigens of the target tissue (Mizuno et al., No Shinkei Geka 20:547-551, 1992; PCT publication WO91/06309; Japanese patent application 1047381; and European patent publication EP-A-43075).

Methods of expressing transgenes in invertebrates (e.g., parasites) are known in the art, for example transfection of enteric parasites (U.S. Pat. No. 5,665,565) and transfection of obligate intracellular parasites (U.S. Pat. No. 5,976,553).

Clones of Transgenic Animals

Clones of the non-human transgenic animals described herein can be produced according to the methods described in Wilmut et al. (Nature 385:810-813, 1997) and PCT publication Nos. WO 97/07668 and WO 97/07669. In brief, a cell, for example, a somatic cell from the transgenic animal, can be isolated and induced to exit the growth cycle and enter the Go phase to become quiescent. The quiescent cell can then be fused, for example, through the use of electrical pulses, to an enucleated oocyte from an animal of the same species from which the quiescent cell is isolated. The reconstructed oocyte is then cultured such that it develops into a morula or blastocyte and is then transferred to a pseudopregnant female foster animal. Offspring borne of this female foster animal will be clones of the animal from which the cell, for example, the somatic cell, was isolated.

Once the transgenic animal is produced, cells of the transgenic animal and cells from a control animal are screened to determine the presence of an RdRP coding sequence using, for example, polymerase chain reaction (PCR). Alternatively, the cells can be screened to determine if the RdRP is expressed (e.g., by standard procedures such as Western blot analysis).

The transgenic animals of the present invention can be homozygous or heterozygous and both can support adenovirus infection. The present invention provides for transgenic animals that carry a transgene of the invention in all their cells, as well as animals that carry a transgene in some, but not all of their cells. That is, the invention provides for mosaic animals. The transgene can be integrated as a single transgene or in concatamers, e.g., head-to-head tandems or head-to-tail tandems.

For a review of techniques that can be used to generate and assess transgenic animals, skilled artisans can consult Gordon (Intl. Rev. Cytol. 115:171-229, 1989), and may obtain additional guidance from, for example: Hogan et al., supra; Krimpenfort et al., Bio/Technology 9:86, 1991; Palmiter et al., Cell 41:343, 1985; Kraemer et al., Genetic Manipulation of the Early Mammalian Embryo, Cold Spring Harbor Laboratory Press, Cold Spring Harbor, N.Y., 1985; Hammer et al., Nature 315:680, 1985; Purcel et al., Science 244:1281, 1986; U.S. Pat. No. 5,175,385; and U.S. Pat. No. 5,175,384.

### EXAMPLES

The invention is further described in the following examples, which do not limit the scope of the invention described in the claims.

EGO-1 and RRF-1 are essential for RNAi in C. elegans. Furthermore, neither of these genes nor their products have been detected in other eukaryotes. The following Examples demonstrate some of these discoveries and provide methods for carrying out the invention.

Prior work demonstrated that EGO-1 activity was necessary only for RNAi targeting in a small subset of the genes tested (i.e., in the germ line). Therefore, there was no evidence for a general role for RdRPs in RNAi. The invention is related to the discovery that RdRPs are essential for all RNAi in C. elegans, and the reason for the limited role for EGO-1 in C. elegans RNAi is the existence of a redundant factor RRF-1.

Prior art would suggest that RdRPs derived from plants or fungi could be used as tools for genetic interference where the initiator is not already double-stranded RNA (e.g., a transgene as an initiator as in co-suppression). These enzymes make dsRNA from a single-stranded mRNA template and thus, as stated above, would not be expected to be essential for RNAi where the inducer is dsRNA provided directly. Indeed, in plants infected with a dsRNA virus that carries its own polymerase, the cellular RdRP enzyme is not necessary for gene silencing. Thus, the novel aspect of this invention is the recognition that members of this RdRP family are essential for genetic interference even when dsRNA is used as the trigger.

One explanation for these findings is that not all dsRNAs are identical and that unique features of dsRNAs formed through RdRP action stimulate interference.

In systems lacking RdRP activity, RNAi is carried out by a small RNA species of approximately 21-26 nucleotides called siRNAs. These siRNAs are formed in vivo by the RNAse III type enzyme Dicer (Bernstein et al., 2001, Nature 409:363-366), or can be constructed in vitro by RNA synthesis using methods known in the art. Introducing siRNAs directly into cells including human cultured cells is sufficient to induce RNAi. The level of RNAi induced by the direct application of siRNAs is likely to be much lower than the level that can be achieved if the siRNAs are subject to amplification by RdRP. When RdRP activity is present in a cell, the targeted mRNA or the siRNAs themselves serve as a templates for the expression of more siRNAs that then go on to catalytically destroy more target mRNAs than in the absence of RdPP activity. The result is a logarithmic expansion of the siRNA species and a much greater level of RNA destruction than can be achieved by application of siRNAs alone. Indeed, in C. elegans the level of RNA destruction achieved by siRNAs alone is below the sensitivity for detection in most RNAi assays used in this organism.

### Example 1

### Experimental Procedures

Strains, Genetic Analysis and Mapping.

The Bristol strain N2 was used as a standard wild-type strain. rde-4 was previously mapped to chromosome III near unc-69 (Tabara et al., 1999, Cell 99:123-132). rde-9 was linked to chromosome I using SNPs that differ between N2 and a wild isolate from Hawaii (Wicks et al., 2001, Nat. Genet. 28:160-164). Genetic mapping placed rde-9 to the left end of the interval between unc-13 and lin-11. Subsequently, unc-13 rde-9/spe-4 lin-10 heterozygous animals were used to generate 16 unc-13 lin-10 recombinants that were assayed for sensitivity to let-2 dsRNA. Of these, six were sensitive to let-2 (RNAi) (unc-13 lin-10), while 10 were resistant (unc-13 rde-9lin-10). The rde-9(ne734) mutation was placed over the chromosome I deficiency ozDf5 by crossing rde-9/unc-13 rde-9 males to unc-13 ozDf5; nDp4 hermaphrodites and picking Unc progeny. F1 rde-9/ozDf5 animals were resistant to let-2 RNAi. Complementation between rde-9(ne734) and ego-1(om71) was tested by mating individual ego-1 unc-29/+ males with unc-13 rde-9 lin-10 hermaphrodites and unc-13 rde-9/ego-1 unc-29 progeny were identified. Fertile non-Unc animals were tested for sensitivity to let-2 (RNAi). Rescue experiments were performed by co-injection of the rescuing construct with a mixture of plasmids that express sense and antisense unc-22 RNA as well as the rol-6 transformation marker (Tabara et al., 1999, supra).

RNA Interference Assays

Synthesis and injection of dsRNA were performed essentially as described (Fire et al., 1998, Nature 391:806-811). N2 and rde-9 hermaphrodites were injected with dsRNA (1-7 mg/ml) targeting the indicated gene. After a period of recovery, injected animals were transferred to fresh plates and allowed to lay eggs for 16 to 24 hours before being removed. The F1 progeny were scored for the expected RNAi phenotypes (Fire et al., 1998; Gonczy et al., 2000, Nature 408:331-336; Maeda et al., 2001, Curr. Biol. 11:171-176; Tabara et al., 1999, supra). Transgenic pes-10::gfp animals were plated as newly hatched L1 larvae onto bacterial lawns expressing gfp dsRNA and assayed as adults for GFP expression under ultraviolet light using a dissecting microscope. The rde-9 mutant was fully sensitive to pos-1 RNAi by feeding, indicating that rde-9 is not resistant to RNAi by feeding.

Immunoprecipitation

RNAi was induced in large populations of worms (50,000 to 100,000) by feeding with E. coli expressing dsRNA as described in Tabara et al., 1999 (supra). The worms were homogenized in lysis buffer (25 mM HEPES-NaOH pH 7.4, 150 mM NaCl, 0.2 mM DTT, 10% glycerol, 1% Triton™ X-100 and complete protease inhibitors (Roche)). For RNA coprecipitation experiments, 2% Superasein (Ambion) was included in the lysis buffer. Affinity-purified anti-RDE-4 antibody, anti-HA antibody 3F10 (Roche) and anti-FLAG antibody M2 (Sigma) were used for immunoprecipitation and immunoblotting. To recover RNA from RDE-4 immunoprecipitates, the precipitates were incubated in 0.2 mg/ml Proteinase K, 0.7×TBS, 5 mM EDTA and 0.1% SDS for 30 minutes at 50° C., and were extracted with phenol and chloroform. Nucleic acids extracted from RDE-4 immunoprecipitates were analyzed by Northern blot analysis after separation on agarose/formaldehyde or polyacrylamide/urea gels essentially as described below.

Northern Blot Analysis

For RNA analyses, worms exposed to gfp dsRNA by feeding were washed in M9 buffer and homogenized in the presence of TRI REAGENT (MRC, Inc.). RNA was extracted from homogenates according to the manufacturers specifications. RNA preparations were quantified and the integrity assessed on 1% agarose gels. To detect high molecular weight RNA species, 20 μg of total RNA was fractionated on agarose/formaldehyde gels. RNA immobilized on Hybond N+ (Amersham) was detected using strand-specific riboprobes. The blots were washed twice with 2×SSC/1% SDS at 50° C. and twice with 0.2×SSC/0.1% SDS at 60° C.

To detect siRNAs, 100 μg of denatured total RNA was resolved on 15% polyacrylamide/6 M urea gels. RNA was transferred to Hybond N+ in 0.5×TBE at 400 mA for one hour using a Trans-blot semi-dry apparatus (BioRad). RNA was immobilized using a UV crosslinker (Stratagene). ³²P-labeled strand-specific riboprobes were synthesized using T7 RNA polymerase and α-³²P-ATP (6000 Ci/mmole; 40 mCi/ml; ICN). After synthesis, probes were partially hydrolyzed in 80 mM NaHCO3, 160 mM Na₂CO₃ at 60° C. for one hour. Hybridization was performed at 50° C. in 45% formamide, 2×SSC, 1×Denhardt's solution, 50 mM Na phosphate (pH 7.2), 7% SDS, and 250 μg/ml denatured salmon sperm DNA. siRNA blots were washed twice with 2×SSC/5% SDS buffer followed by several washes in 1×SSC/1% SDS at 50° C. Autoradiography was performed using film or by exposing blots to phosphoimaging screens (Fuji) and analyzing images using a BioRad phosphoimager and QuantityOne software (BioRad). Synthetic 22nt gfp RNA oligonucleotides (Dharmacon Research Inc.) were used as hybridization controls (1 pmole/lane).

Heat-shock Assay.

hsp16-2::gfp transgenic lines were made by transformation of pCMM317 construct provided by Yingdee Unhavaithaya along with the pRF4 transformation marker. The pes-10::gfp integrated strain JH103 was provided by Geraldine Seydoux. It was crossed with hsp16-2::gfp to obtain hsp16-2::gfp; pes-10::gfp strain. hsp16-2::gfp, pes-10::gfp and hsp16-2::gfp; pes-10::gfp worms were cultured starting from L2-L3 for 48 h on bacteria expressing gfp dsRNA. Control populations of the same stage were cultured under regular conditions. pes-10::GFP expression in worms exposed to dsRNA was monitored and was completely silent by the end of the 48 hour period. Then gfp dsRNA fed strains and controls were heat-shocked for 4 hours at 33° C. and hsp16-2::GFP expression was scored using fluorescent microscopy (Zeiss).

### Example 2

### Identification of Genes Affecting RNAi

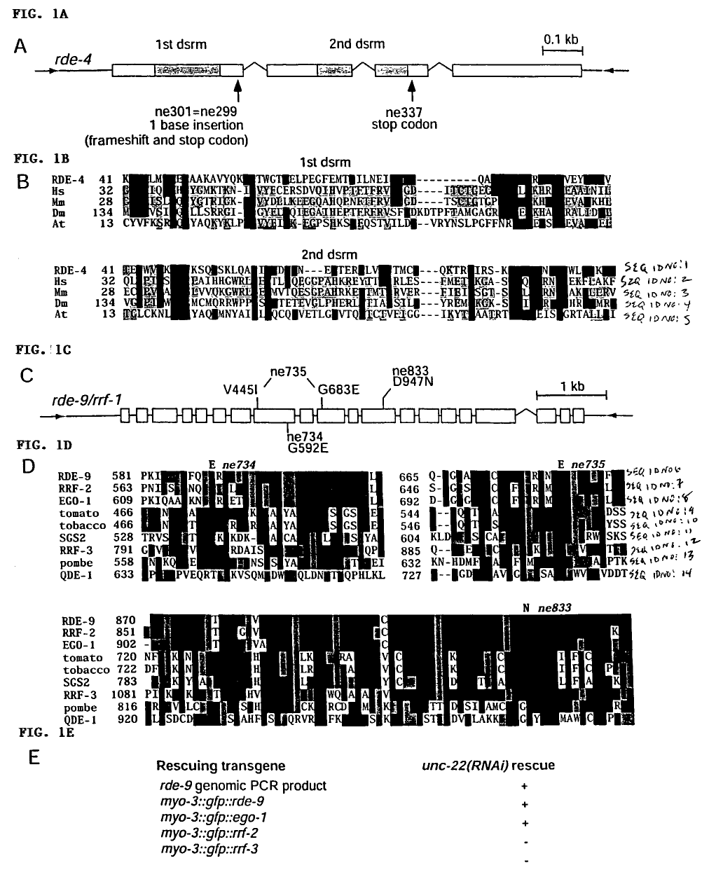

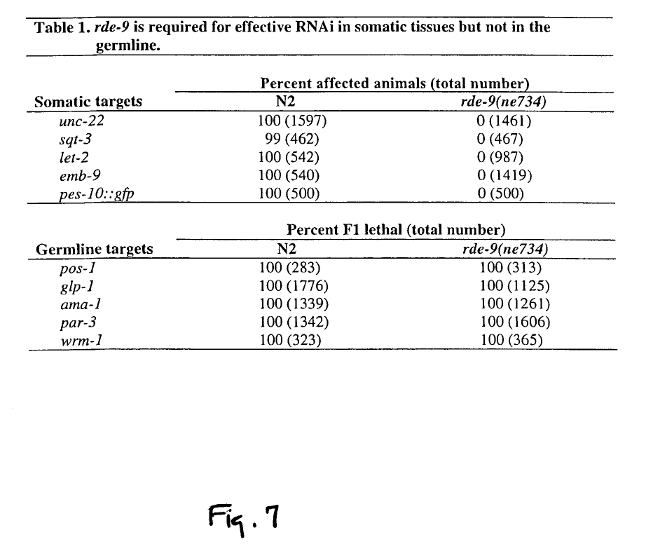

To identify genes affecting RNAi, large populations of mutagenized C. elegans were exposed to RNAi targeting the muscle-specific gene unc-22. The unc-22 gene provides a sensitive assay for somatic RNAi. Promoter-driven synthesis of unc-22 dsRNA causes the distinctive unc-22 twitching phenotype and nearly total body paralysis. Among existing rde mutants only mutations in rde-1 and rde-4 fully eliminate interference in response to promoter driven unc-22 RNAi. Because six alleles of rde-1 already exist, the starting strain was engineered to express extra copies of rde-1(+) activity. Using this strategy, two new alleles of rde-4 as well as five new mutants that define at least one new locus, rde-9, were identified (FIG. 1). Interestingly, rde-9 mutants were resistant to RNAi for genes expressed in somatic tissues, but were fully sensitive to dsRNA targeting genes expressed in the germline (FIG. 7).

The rde-9 mutations mapped to chromosome I within a small genetic interval defined by 4 fully sequenced cosmid clones (FIG. 1A). Rescue of the rde-9 mutant phenotype was achieved by injection of cosmid F26A3, which contains, in addition to several other genes, ego-1 and rrf-1, two of the four C. elegans RdRP related genes. Using PCR primers designed to amplify individual genes from this region, rde-9 rescue was found to map to the rrf-1 locus (FIG. 1A). Genomic sequencing of the three rde-9 alleles identified unique point mutations in each allele that alter highly conserved amino acid residues in the predicted rrf-1 open reading frame (FIG. 1A). Surprisingly, the rde-9 (ne735) allele exhibited two mutations (FIG. 1A). The three rde-9 alleles behave as simple recessive loss of function mutations and exhibit similar complete resistance to somatic RNAi. The rde-9 (ne734) allele behaved in a manner expected for a genetic null allele when placed over a chromosomal deficiency. Finally, consistent with the idea that the alterations in rrf-1 and not ego-1, are responsible for the rde-9 phenotype, rde-9 alleles were found to complement ego-1 (om71) producing viable and fertile animals that are sensitive to RNAi (22). These findings demonstrate that rrf-1 and ego-1 define distinct genes and that rde-9 mutations represent loss of function alleles of rrf-1. Pending approval of the C. elegans Genetics Center and other C. elegans researchers, the rrf-1 gene will be renamed rde-9 to reflect its function in RNAi.

Ego-1 was previously shown to be important for fertility and for RNAi targeting certain germline expressed genes, but was dispensable for RNAi targeting somatic genes. Since rde-9 mutants have a reciprocal profile, resistance to RNAi in the soma and sensitivity in the germline, rde-9 and ego-1 could encode functionally equivalent but tissue-specific factors required for RNAi. To address this question, coding sequences from ego-1, rde-9 as well as the other two C. elegans RdRP family members, rrf-2 and rrf-3, were expressed under the muscle-specific myo-3 promoter, and were tested in rde-9 (ne734) mutant animals for rescue of RNAi targeting unc-22. Both the rde-9 and ego-1 constructs exhibited strong rescue in this assay while the rrf-2 and rrf-3 constructs failed to rescue (FIG. 1B). Thus, rde-9 and ego-1 appear to represent tissue-specific, but otherwise functionally equivalent factors essential for RNAi.

These data show that rde-9 and ego-1 can be used interchangeably in systems (e.g., therapeutic) in which RNAi is enhanced by increasing RdRP activity.

### Example 3

### Rde-9 is Necessary for Accumulation of Anti-sense RNA in C. elegans

The requirement for an RdRP related protein in RNAi is surprising. In the screen for rde-9 mutants, the unc-22 dsRNA was expressed directly in the muscle via a muscle-specific promoter, providing in a continuous source of trigger dsRNA. In addition, large quantities of the trigger dsRNA are used for injection. Thus, it seems unlikely that RDE-9 would be required for the synthesis of the dsRNA that triggers RNAi. Perhaps RDE-9 and by inference other memebers of the RdRP family have some novel function in RNAi other than RdRP activity. Another, possibility is that RdRP activity is involved in the amplification of an RNA species that functions at some later step in RNAi. Consistent with such a model, Grishok et al.,2001, Cell 106:23-34, have found that RNAi in C. elegans is correlated with specific accumulation of an antisense siRNA species. A low level of symmetric processing of the trigger dsRNA into 22 nt fragments has been observed previously. However, Grishok et al. (supra) found that a remarkable accumulation, specifically of the antisense siRNA species occurs during RNAi but only if the target mRNA is present. This accumulation could reflect retention of siRNAs that succeed in destroying an mRNA, but might also reflect an amplification of the successful siRNA species.

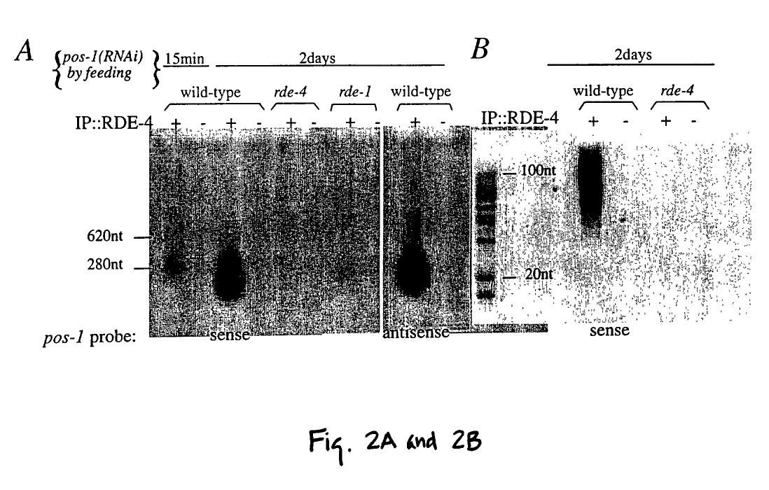

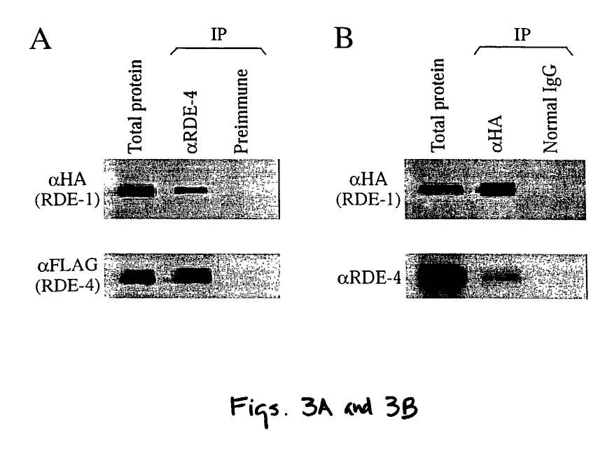

To determine whetehr rde-9 is necessary for the target-dependent accumulation of antisense siRNAs, wild-type and rde-9 mutant animals bearing a GFP transgene were cultured on E. coli expressing GFP dsRNA. RNA prepared from these animals and from control populations were then examined for antisense siRNA accumulation. Accumulation of antisense siRNA failed to occur in rde-9 mutants (FIG. 2). This finding is consistent with a role for RDE-9 in amplification of the antisense siRNA species (FIG. 3). However, any factor required for the initial targeting of the mRNA should in theory exhibit this phenotype. Indeed, the rde-1(+) and rde-4(+) activities are also required for the target dependent accumulation of antisense siRNA (FIG. 2).

Two possible models are presented in which RdRP activity could play a role in the target-dependent amplification of the antisense siRNAs (FIG. 3). In both models, dsRNA processing by the Dicer complex results in a pool of duplex siRNAs that enter a second complex, the targeting complex, which unwinds the siRNAs to permit pairing with the target mRNA. In model A, the mRNA or fragments derived from it, serve as templates for synthesis of more of the antisense strand. The resulting dsRNA could then be processed by Dicer, or alternatively, the newly synthesized antisense RNA might be liberated from the target RNA allowing it to directly complex with the RNAi silencing complex (RISC) to target additional mRNAs. In model B, the targeting complex engages the antisense siRNA with the target and the sense siRNA serves as a template for rounds of antisense siRNA synthesis. These antisense siRNAs then enter the RISC complex, which targets mRNA destruction. These models lead to different predictions. For example, siRNAs derived from sequences upstream of the targeted region should be generated in model A, while such a “spreading” effect would not take place in model B.

Members of the RDE-9 family of RdRPs are absent from the sequenced genomes of other animal species, including Drosophila and humans. Consistent with this finding, Drosophila cell extracts efficiently process dsRNA into 22 nt fragments, while in C. elegans in vivo, this direct processing reaction appears to be less efficient. In plants, the genomes of which contain RdRP, co-suppression has been shown to be associated with a symmetric accumulation of siRNA species, suggesting that the contributions of direct processing versus amplification may differ in different species or in different PTGS mechanisms.

Strategies for intervening in post-transcriptional gene silencing include methods for blocking silencing factors when silencing is deleterious or for providing or enhancing silencing factors when their activities might prove beneficial. Introducing RDE-9 or related proteins could dramatically enhance RNAi as a tool for probing gene function in cells or organisms that might otherwise lack their activities.

Without committing to any particular theory, RdRP gene products may serve to amplify the dsRNA or small interfering RNAs (siRNAs) that mediate RNAi.

### Example 4

### RDE-4 Binds to dsRNA and to RDE-1 in vivo

RDE-4 contains two copies of a motif found in several dsRNA binding proteins, and as expected, it was found that recombinant RDE-4 binds to dsRNA in vitro and that this binding was efficiently competed by dsRNA, but not by ssRNA. The question of whether RDE-4 interacts with dsRNA during RNAi in vivo was investigated. To address this question, polyclonal rabbit antibodies were raised against the recombinant RDE-4 protein. Lysates were prepared from animals that were cultured on bacteria expressing a dsRNA sequence corresponding to the full-length pos-1 cDNA. RDE-4 was immunoprecipitated from the lysates using the anti-RDE-4 antibody, the precipitate was extracted to purify any associated RNA, then analyzed by agarose and acrylamide gel electrophoresis. Northern blot analysis using pos-1 sense and antisense radiolabeled RNA probes detected approximately equal amounts of pos-1 RNA (FIG. 2A). The bulk of this RNA species migrated below 300 nucleotides on an agarose gel (FIG. 2A) and between 70 and 140 nucleotides on an acrylamide gel (FIG. 2B). Little or no pos-1 RNA co-immunoprecipitated with RDE-4 purified from populations that were either not exposed to dsRNA or exposed for only 15 minutes (FIG. 2A and data not shown).

The probes used in the above experiments were designed to detect sequences that are present in the trigger dsRNA. To determine if pos-1 mRNA sequences might also co-immunoprecipitate with RDE-4, we probed for 3′UTR sequences that are not present in the dsRNA trigger. These experiments failed to detect any co-precipitating RNA corresponding to the 3′UTR portion of the pos-1 mRNA. Thus RDE-4 is likely to interact either with the introduced dsRNA itself or with an intermediate RNA species derived from the targeted region.

Previous genetic data suggested that rde-1 and rde-4 are important for the initiation of RNAi (Grishok et al., 2000, Science 287:2494-2497). Therefore, the question of whether the interaction between RDE-4 and RNA in vivo requires the activity of RDE-1 was investigated. Extracts were prepared from rde-1 mutants cultured on pos-1 dsRNA. RDE-4 was immunoprecipitated from these extracts and assayed for co-precipitation of pos-1 RNA. Strikingly, only trace amounts of pos-1 RNA were detected in the precipitate from the rde-1 mutant strain (FIG. 2A), suggesting that RDE-1 activity is required for RDE-4 to engage pos-1 dsRNA in vivo.

To determine whether RDE-4 forms a complex with RDE-1 in vivo, we generated a transgenic strain expressing RDE-1 tagged with the HA epitope and RDE-4 tagged at the carboxy terminus with the FLAG epitope (see Experimental Procedures). The tagged proteins were functional and able to rescue the corresponding mutants. RDE-4 and RDE-1 proteins were immunoprecipitated via the tag sequences and the precipitates were analyzed by immunoblotting. In reciprocal assays, RDE-1 and RDE-4 were found to co-precipitate (FIG. 3). The interaction between RDE-1 and RDE-4 (FIG. 3) occurred in animals that were not exposed to an introduced trigger dsRNA. Therefore, RdPP can enhance RNAi in which endogenous dsRNA is present. A target-dependent step in RNAi requires RDE-1 but not RDE-4 or RDE-9.

Previous attempts to follow accumulations of RNA sequences during RNAi suggested that both the trigger dsRNA and the target mRNA were rapidly degraded in wild-type animals (Montgomery et al., 1998, Proc. Nat. Acad. Sci. USA 95:15502-15507). Experiments were performed to determine whether RNA processing is impaired in rde-mutant strains, perhaps allowing observation of what would otherwise be transient RNA species. In order to identify RNA intermediates that depend on mRNA targeting, animals were exposed to dsRNA corresponding to an exogenous gene. For this assay, animals on bacteria expressing a trigger dsRNA corresponding to the jellyfish green fluorescent protein (GFP) encoding gene.

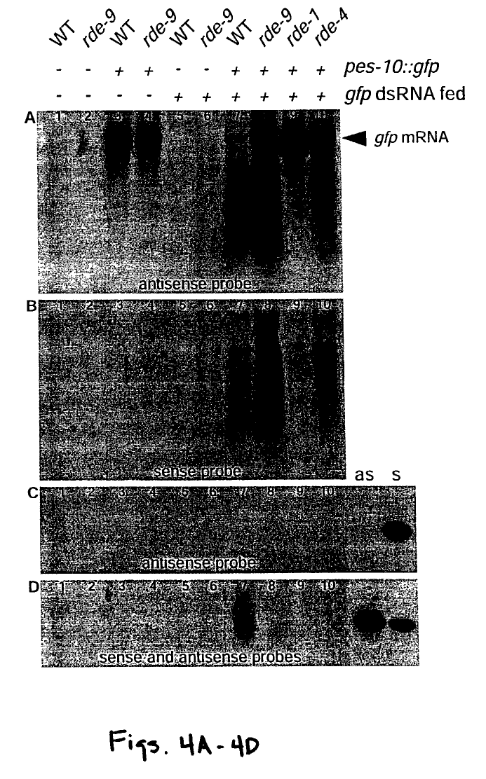

GFP-transgenic animals that were not exposed to the GFP dsRNA exhibited robust expression of GFP sense RNA (FIG. 4A, lane 3). In contrast, non-transgenic animals failed to exhibit any significant hybridization to either antisense or sense RNA probes (FIGS. 4A and 4B, lanes 1 and 5). Furthermore, even after constant feeding on bacteria expressing GFP dsRNA, the non-transgenic strains, including both wild-type and rde-9 mutant strains, failed to exhibit detectable GFP sense and antisense RNA species (FIGS. 4A and 4B, lanes 5 and 6).

A very different result was obtained when animals that carry a target GFP-transgene were exposed to GFP dsRNA. As expected, the abundance of the GFP sense transcript was dramatically reduced in wild-type transgenic animals exposed to GFP dsRNA (FIG. 4A, lane 7). However, while the full-length mRNA was diminished, a marked accumulation was observed of complementary RNAs (cRNAs) consisting of both sense and antisense RNA strands and ranging in size from approximately 100 nt to the full length of the GFP transcript (FIGS. 4A and 4B, lane 7). To further investigate the nature of the cRNA species, the expression and accumulation of cRNA and GFP mRNA species in rde-1, rde-4 and rde-9 mutants was examined. As expected, transgenic versions of each of these mutant strains exhibited wild-type levels of GFP mRNA expression, even when cultured in the presence of GFP dsRNA trigger (FIGS. 4A and 4B, compare lanes 3 and 4 to lanes 8, 9, and 10). Strikingly, however, cRNA species accumulated to very high levels in rde-9 mutant animals and also appeared to be enriched relative to wild-type in rde-4 mutant animals (FIGS. 4A and 4B, compare lanes 8 and 10 to lane 7). In contrast, rde-1 mutants exhibited, at most, a slight accumulation of cRNA (FIGS. 4A and 4B, lane 9). Thus, RDE-1 activity, a target gene and a trigger dsRNA are all required for cRNA accumulation. In contrast, RDE-4 and RDE-9 activities are not required for cRNA accumulation. Instead, removing their activities either accelerates cRNA accumulation or blocks its further processing.

The accumulation of another RNA species, the previously described small interfering, siRNAs was examined. For these experiments, polyacrylamide gel electrophoresis was used to resolve the small RNA species in each RNA population (FIG. 4, panels C and D). Whereas both rde-9 and rde-4 mutants exhibited accumulation of higher molecular weight cRNA sequences, both of these mutants, as well as rde-1 mutants, failed to exhibit siRNA accumulation (FIGS. 4C and 4D, lanes 8, 9, and 10). Only wild-type animals undergoing GFP RNAi exhibited siRNA accumulation (FIG. 4D, lane 7). Furthermore, whereas both sense and antisense siRNAs have been associated with PTGS in both plants and Drosophila, we found that only the antisense siRNA species accumulated in these experiments (FIG. 4, lane 7, compare panels C and D).

### Example 5

### Target-dependent Silencing

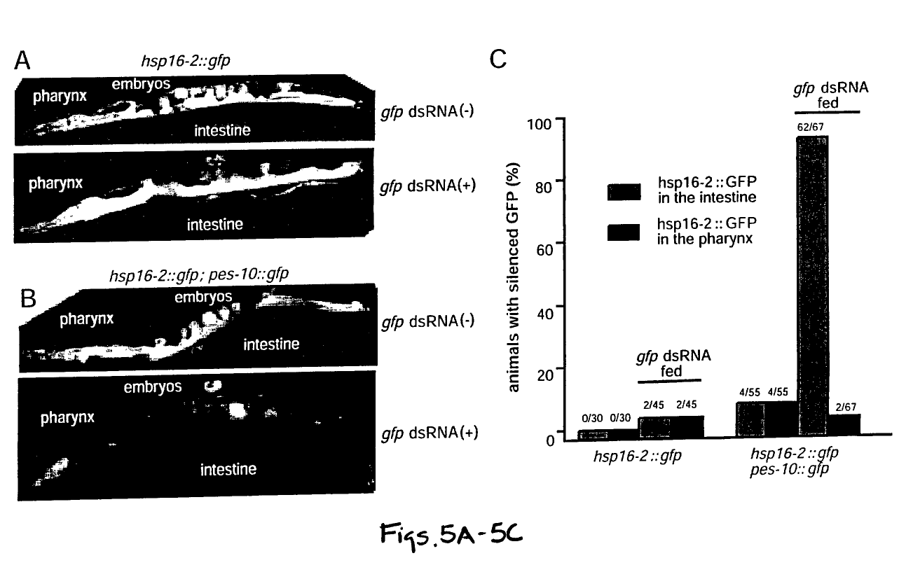

The above studies suggest that a target-dependent event leads to retention or amplification of active siRNA species. The question was then examined of whether this target-dependent process facilitates the response to future challenge. To examine this possibility, a strain was created that carries a GFP driven from the tightly inducible heat-shock promoter (hs::gfp, see Experimental Procedures). This strain was crossed to generate animals that carry both the inducible hs::gfp transgene and a second constitutive GFP transgene (pes-10:: gfp). Worm populations carrying the hs::gfp transgene, with or without the constitutive pes-10::gfp transgene, were cultured in the presence of dsRNA trigger and after 48 hours were heat shocked to induce expression of the hs::gfp transgene. Animals lacking the constitutive pes-10::gfp transgene exhibited robust hs::GFP expression in their pharynx and intestine whether or not they were exposed to gfp dsRNA (FIGS. 5A and C). Thus dsRNA feeding in the absence of a target mRNA failed to prevent expression of the hs::GFP transgene. Animals carrying the constitutive pes-10::gfp transgene that were not exposed to dsRNA exhibited bright pes-10::GFP fluorescence in their intestine and upon heat shock exhibited additional GFP expression in the intestine, as well as intense hs::GFP expression in the pharynx (FIG. 5B, upper panel). In contrast, animals undergoing silencing of the pes-10::gfp transgene exhibited a mosaic pattern of hs::GFP expression. In these animals, hs::GFP was not present in the intestine but was still expressed at high levels in the pharynx and embryos (FIG. 5B, lower panel, and FIG. 5C). Thus the induction of hs::gfp was prevented only in the intestine, the tissue where the pes-10::gfp was already undergoing silencing. These findings suggest that exposure to dsRNA alone is not sufficient to induce silencing, but requires concomitant expression of the target gene.

In summary, the above examples show that two steps have been identified in RNAi that depend on the presence of a complementary target sequence. The first of these events requires the activity of the RNAi pathway gene rde-1 and leads to the accumulation of an RNA species we have termed complementary RNA (or cRNA). The cRNA species consists of sense and antisense RNAs, possibly dsRNAs, that range in size from 100 nt to the full-length of the target RNA sequence. A second event requires rde-1 and two additional genes, rde-4 and rde-9, and leads to the accumulation of antisense siRNA. It is shown herein that RDE-4 is a dsRNA binding protein and that RDE-9 is one of four C. elegans homologs of plant and fungal RNA-dependent RNA polymerase related proteins. Finally it is shown herein that the target-dependent process enhances a localized silencing effect.

C. elegans RdRPs have evolved unique functional domains through which they interact with proteins that mediate RNAi. RdRPs and these domains are absent from many other animals including Drosophila and humans. Therefore, even though other RNAi components are present in these organisms lacking the unique RdRP functional domains, the RNAi proteins may lack domains necessary for interacting with RdRP. Therefore, to increase RNAi activity, in some cases, that not only the C. elegans RdRP but also other RNAi mediators that can interface with these RdRP enzymes may be needed for efficient RNAi. Transfer of rrf-1, ego-1, and other C. elegans genes to other organisms can be accomplished using well-established techniques known to one educated in the art of molecular biology.

### OTHER EMBODIMENTS

A number of embodiments of the invention have been described. Nevertheless, it will be understood that various modifications may be made without departing from the spirit and scope of the invention. For example, it is possible that the RdRP-related proteins EGO-1 and RRF-1 (also known as Rde-9) have functions in addition to the RNA polymerase function. Providing or activating these activities may also enhance RNAi. Accordingly, other embodiments are within the scope of the following claims.

What is claimed is:

1. A method for inducing or enhancing RNA interference (RNAi) in a cell, the method comprising
(a) obtaining a cell, and
(b) increasing the expression or activity of an RNA-dependent RNA polymerase (RdRP) in the cell.

2. The method of claim 1, wherein the expression or activity of an RdRP is increased by providing an RdRP to the cell.

3. The method of claim 2, wherein the RdRP is provided to the cell by transfecting the cell with an RdRP-expressing gene construct.

4. The method of claim 1, wherein the RdRP is an EGO-1 or RRF-1.

5. The method of claim 1, wherein the RdRP is a C. elegans RdRP.

6. The method of claim 1, wherein the cell is a mammalian cell.

7. The method of claim 1, wherein the RdRP is a C. elegans RdRP and the cell is a mammalian cell.

8. The method of claim 1, wherein the RdRP is an RdRP of an invertebrate animal and the cell is a cell of a vertebrate.

9. The method of claim 1, wherein the expression or activity of RDE-1, RDE-4, or DCR-1 is increased in the cell.

10. The method of claim 1, further comprising introducing an siRNA into the cell.

11. The method of claim 2, further comprising introducing an siRNA into the cell.

12. A vector comprising a nucleic acid molecule encoding an RdRP.

13. The vector of claim 12, wherein the nucleic acid molecule comprises ego-1 or rrf-1.

14. The vector of claim 12, wherein the ego-1 or rrf-1 is operatively linked to a mammalian regulatory sequence.

15. A mammalian cell comprising the vector of claim 12.

16. The mammalian cell of claim 15, wherein the cell is a non-human cell.

17. A transgenic non-human mammal that expresses an ego-1 or rrf-1 sequence.
